(19) United States
(12) Patent Application Publication
(10) Pub. No.: US 2017/0281236 A1
(43) Pub. Date: Oct. 5, 2017

(54) EXTERNAL FIXATION CLAMP

(71) Applicant: MIKAI S.P.A., Genova (IT)

(72) Inventor: Nicola MUSSOLIN, Genova (IT)

(21) Appl. No.: 15/507,551

(22) PCT Filed: Sep. 4, 2015
(86) PCT No.: PCT/IB2015/056772

§ 371 Date: Feb. 28, 2017

(30) Foreign Application Priority Data
Sep. 4, 2014 (IT) BO2014A000484

(51) Int. Cl. A61B 17/64; A44B 99/00

(52) U.S. Cl. CPC A61B 17/645; A44B 99/00

(57) ABSTRACT

External fixing system (1) for orthopaedic exoskeleton; the fixing system (1) comprises two clamps (2, 6), which have clamping seats (5, 9) and respective knurled elements (13, 14) facing each other, and a spring (12) for pushing the clamps (2, 6) one against the other so that their relative rotation is prevented by the coupling of the two knurled elements (13, 14); in this way, in use, it is possible to obtain locking of the relative position of the clamps (2, 6) while the clamps (2, 6) are tightened by means of an Allen screw (23) arranged in an opposite position with respect to the clamping seats (5, 9).

### TECHNICAL FIELD

The present invention relates to an external fixing system for an orthopaedic exoskeleton.

### CONTEXT OF THE INVENTION

Exoskeletons for orthopaedic use usually comprise support elements (for example bars and/or screws) and serve to stabilise a limb after a serious trauma. More precisely, exoskeletons are used to reduce the risk of secondary traumas and to obtain primary stabilisation, giving the patient's body time to recover from the acute phase before a subsequent surgical operation.

One of the most important components of the exoskeletons of the type described above is the fixing system, which is an orthopaedic device able to connect two transverse support elements (screws and/or bars) in a rigid manner.

FIG. 1 illustrates a first example of fixing system A in the state of the art. Said fixing system A comprises a clamp B, which has a first clamping seat C adapted to house a first bar (or screw), and a clamp D, which is adjacent and connected to the clamp B in a rotary manner and has a clamping seat E adapted to house a further bar. A spring is arranged around a connecting rod (partially illustrated) between the clamps B and D to allow a relative rotation of said clamps. In use, it is possible to rotate a knob F to screw-tighten the rod in the clamp D and therefore bring the clamps B and D close to each other so as to lock the relative rotation of said clamps B and D and tighten the seats C and E in order to clamp the bars arranged therein.

The fixing systems of this type have various problems including:
difficulty of closing the clamps, since the application of a high rotation force on the knob F is necessary to obtain secure locking;
when a high force is applied to the knob F, a lever effect is produced on the bars connected to the fixing system and the bars can therefore be easily damaged;
the need to use torque wrenches to know whether the correct force is being applied (excessive force entails the risk of breakage);
the correct relative orientation of the two clamps B and D is difficult to obtain, since due to the interposed spring, the clamps are free to rotate with respect to each other;
the system is not very flexible as it is possible to use only bars and/or screws with specific defined diameters.

FIGS. 2 and 3 illustrate a second example of fixing system A′ in the state of the art. Said fixing system A′ is substantially identical to the fixing system A, from which it differs only due to the fact that to lock the relative movement of the clamps B and D, an Allen screw G is tightened instead of rotating the knob F. The fixing system A′ has fewer problems connected with the lever effect exerted on the bars/screws. However, it has the drawback of making a stable closure difficult, since a high force is required to tighten the Allen screw G.

The object of the present invention is to provide an external fixing system for an orthopaedic exoskeleton, said system overcoming, at least partially, the drawbacks of the known art and at the same time being easy and inexpensive to produce.

### SUMMARY

According to the present invention, an external fixing system is provided as claimed in the following independent claim and, preferably, in any one of the claims depending directly or indirectly on the independent claim.

### BRIEF DESCRIPTION OF THE FIGURES

The invention is described below with reference to the attached figures, which illustrate some non-limiting embodiment examples, in which:

FIG. 1 is a lateral view of a fixing system in the state of the art;

FIG. 2 is a perspective view of a further fixing system in the state of the art;

FIG. 3 is a lateral view of the system of FIG. 2;

FIG. 4 is an exploded perspective view of a fixing system according to the present invention;

FIG. 5 is an exploded perspective view of a further embodiment of the fixing system according to the present invention;

FIG. 6 is a lateral view of a further embodiment of the fixing system according to the present invention; and

FIG. 7 is an exploded perspective view of a further embodiment of the fixing system according to the present invention.

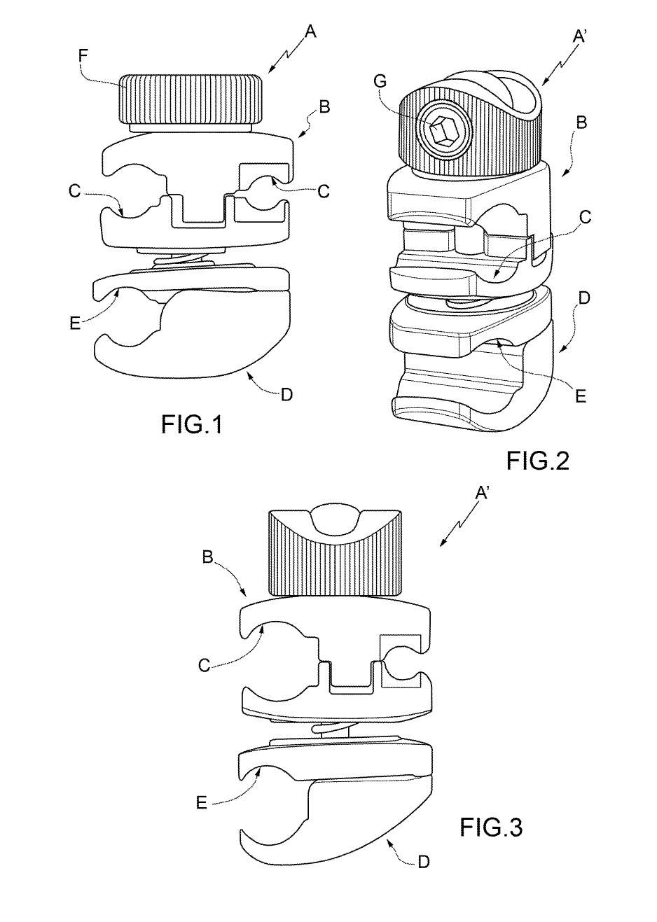

### DETAILED DISCLOSURE

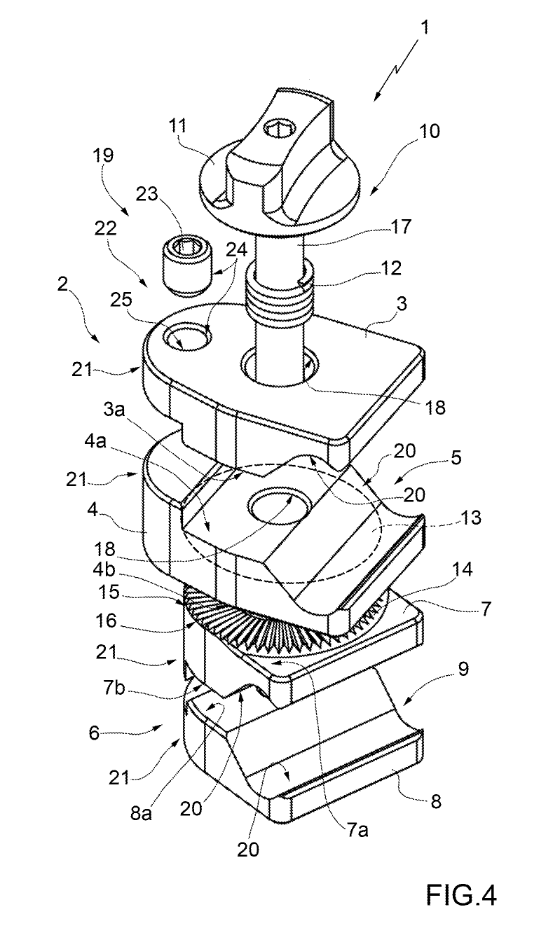

In FIG. 4, the number 1 indicates as a whole an external fixing system for an orthopaedic exoskeleton (known per se and not illustrated), which comprises two support elements (for example, a bar and a screw or two bars).

The fixing system 1 comprises a clamp 2, which has two jaws 3 and 4 and a clamping seat 5 adapted to house a first support element, and a clamp 6, which is adjacent and connected to the clamp 2 (in an orientable and in particular a rotary manner) and has two jaws 7 and 8 and a clamping seat 9 adapted to house a second support element.

According to some embodiments, the system 1 further comprises a locking assembly 10 to limit, in particular lock, the relative orientation (in particular, rotation) of the clamps 2 and 6 with respect to each other. The locking assembly 10 comprises a supporting element 11, which is arranged on the opposite side of the clamp 2 with respect to the clamp 6 (in other words, the clamp 2 is arranged between the supporting element 11 and the clamp 6) and is connected to the clamp 6, and an elastic element 12 arranged (in particular, in contact) between the supporting element 11 and the clamp 2 (in particular, the jaw 3) to exert a force on the clamp 2 towards the clamp 6.

It should be noted that the particular position of the elastic element 12 allows the clamps 2 and 6 to be kept in contact at rest (i.e. when external forces are not applied to the system 1), stabilizing (at least partially) their relative orientation.

More precisely, in use, the operator (the surgeon) selects the most appropriate relative orientation of the clamps 2 and 6 for the operation to be performed. At this point, he can carry out other work (for example, tightening of the seats 5 and 9) in relative tranquillity and with a very low risk of the relative position of the clamps 2 and 6 varying. In this way, particularly precise operations can be performed on the patients' bodies.

According to some embodiments, the locking assembly 10 comprises at least one locking element 13 (illustrated by a broken line) arranged on the clamp 2, and a locking element 14, which is arranged on the clamp 6 and is adapted to couple with the locking element 13 to limit (more precisely, to lock) the relative rotation of the clamps 2 and 6 with respect to each other.

More precisely, the locking element 13 is arranged on the jaw and the locking element 14 is arranged on the jaw 7. The jaws 4 and 7 face each other and are arranged between the jaws 3 and 8.

According to some embodiments, the locking elements 13 and 14 have respective shapes substantially complementary to each other. In particular, at least one of the locking elements 13 and 14 has at least one projection 15. At least the other of the locking elements 13 and 14 has at least one seat (one depression) 16 adapted to be engaged by the projection 15 to limit, in particular to lock, the relative rotation of the clamps 2 and 6 with respect to each other.

Advantageously, the locking elements 13 and 14 each have a plurality of projections 15 and a plurality of seats 16 which are (adapted to be) engaged by the projections 15. According to the embodiment illustrated, the projections 15 extend radially with respect to a circumference defined by a succession of said projections 15.

In particular, the locking elements 13 and 14 are knurled.

According to some embodiments, the system 1 also comprises a connecting rod 17 (cylindrical with circular section), which is connected to the clamp 6 and (integrally) to the supporting element 11 and extends through the clamp 2. The first clamp 2 is (at least partially) able to slide along the rod 17.

Advantageously, the spring (of the elastic element 12) is arranged around the rod 17.

More precisely, the connecting rod 17 extends through holes 18 (through holes) of the jaws 3, 4, 7 and is connected (in a non-inclinable manner) to the jaw 8. In particular, the holes 18 have a substantially circular shape.

The locking assembly 13 comprises a coupling system (not illustrated) between the rod 17 and the clamp 6 (in particular, the jaw 8), said coupling system being adapted to normally keep the distance between the supporting element 11 and the clamp 6 fixed (in other words, the coupling system fixes the rod 17 to the clamp 6) and is adapted to be activated to vary said distance.

In particular, the coupling system comprises two threads, which are adapted to cooperate to allow the supporting element 11 to move closer to or farther away from the clamp 6. More precisely, one of the threads is arranged in the clamp 6 (in particular, in the jaw 8) and the second thread is arranged on the rod 17 (in particular, in the area of one end of the rod 17 opposite the supporting element 11).

In the embodiment illustrated, the supporting element 11 is provided with a knob which can be used by the operator as a grip for rotating the rod 17.

Advantageously, between the clamps 2 and 6 there are no elastic means (which in resting conditions allow the clamps 2 and 6 to be kept at a distance). Said elastic means (absent) can be of any known type; for example, a rubber element and/or a spring are not interposed between the clamps 2 and 6.

In particular, the clamps 2 and 6 are directly in contact with each other (without the interposition of any element).

According to some embodiments, the system 1 comprises a closing device 19 to vary the width of the clamping seat 5 by modifying the relative orientation (inclination) of the jaws 3 and 4. Advantageously, the closing device 19 is adapted to vary also the width of the clamping seat 5 by modifying the relative orientation (inclination) of the jaws 7 and 8. In particular, each jaw 3, 4, 7 and 8 has a respective locking surface 20. The locking surfaces 20 of the jaws 3 and 4 delimit at least partially the clamping seat 5; the locking surfaces 20 of the jaws 7 and 8 delimit at least partially the clamping seat 9. Each jaw 3, 4, 7 and 8 has one end 21 opposite the respective locking surface 20.

Advantageously, the closing device 19 comprises at least one thrust assembly 22 adapted to exert a thrust which pushes apart said ends 21 of the jaws 3 and 4. In particular, the thrust assembly 22 comprises a thrust element 23 arranged in the area of the ends 21 (more precisely, the end of the jaw 3) to exert a thrust which pushes apart the ends 21 of the jaws 3 and 4 and (consequently) a force which brings the locking surfaces 20 of the first and second jaw 3, 4 closer together.

The closing system 19 offers various advantages with respect to the state of the art, including the following.
The jaws can be tightened by the application of a relatively low force; particularly favourable levers can be exploited with particularly long arms which extend from the end 21 (of the jaws 3 and 7) to the seat 5 and to the seat 9, respectively (with fulcrum in the area of the rod 17). In this regard, it should be noted that in the systems in the state of the art, the lever effect that can be exploited is lower and even negligible.
The risk of damaging the bars or the screws arranged in the seats 5 and 9 is reduced since, during closing of the clamps 2 and 6, a distortion force amplified by a lever effect on said bars and screws is not applied.
Since the jaws 3 and 4 change their relative orientation, the system 1 can be used for bars and screws of different dimensions.

According to some embodiments, the rod 17 is arranged between the locking surfaces 20 and the ends 21.

In particular, the rod 17 is arranged between the locking surfaces 20 and the thrust element 23.

In particular, the clamps 2 and 6 are arranged and shaped so that the pushing-apart thrust exerted by the thrust assembly 22 is transferred to the clamp 6 in order to exert a force on the jaw 7 so that the locking surfaces 20 of the jaws 7 and 8 move closer to each other and the ends 21 of the jaws 7 and 8 move away from each other.

According to some embodiments, the jaw 3 has a coupling surface 3a. The jaw 4 has a coupling surface 4a which faces (in particular in contact with) the coupling surface 3a, and a coupling surface 4b (in particular, opposite the surface 4a). The third jaw 7 has a coupling surface 7a which faces (in particular in contact with) the coupling surface 4b, and a coupling surface 7b (in particular, opposite the surface 7a). The jaw 8 has a coupling surface 8a which faces (in particular in contact with) the coupling surface 7b.

According to some embodiments, the surface 3a has a (slight) convexity (or concavity) facing (in particular, in contact with) the surface 4a; and the surface 4a has a (slight) concavity (or convexity, where the surface 3a has a convexity) facing (in particular, in contact with) the surface 3a.

In addition or alternatively, the surface 7b has a (slight) convexity (or concavity) facing (in particular, in contact with) the surface 8a; and the surface 8a has a (slight) concavity (or convexity, where the surface 7b has a convexity) facing (in particular, in contact with) the surface 7a.

In use, when the thrust assembly 22 is activated, the locking surface 20 of the jaw 3 is pushed towards the surface 20 of the jaw 4 so as to tighten a respective support element interposed between them. In this way, also the end of the jaw 3 opposite the end 21 and, consequently, the end of the jaw 7 opposite the end 21 are pushed towards the jaw 8 (which, it should be noted, is integral—at least as regards the relative inclination—with the rod 17).

According to some embodiments, the thrust element 23 is adapted to project from the jaw 3 towards the jaw 4 so as to come into contact with the jaw 4 (and move the end 21 of the jaw 4 away from the jaw 3).

In particular, the thrust assembly 22 comprises a connection system 24 between the thrust element 23 and the jaw 3, said connection system 24 being adapted to maintain normally integral the thrust element 23 with the jaw 3 and can be activated to vary the position of the thrust element 23, more precisely in the direction of the jaw 4. In other words, in use, it is possible to act on the connection system 24 to vary the extension of the thrust element 23 from the jaw 3 (towards the jaw 4).

Advantageously, the connection system 24 comprises two threads (of per se known type and not illustrated), which are adapted to cooperate to allow the thrust element 23 to move closer to or farther away from the jaw 4, provoking a relative rotation between the thrust element 23 and the first clamp 2. In particular, a thread is obtained on the thrust element 23 and a thread is obtained in the jaw 3.

In other words, the thrust element 23 can be tightened in the jaw 3.

According to the embodiment illustrated, the thrust element 23 is an Allen screw adapted to be screw-tightened into a through hole 25 (threaded) of the jaw 3.

In particular, the connection rod 17 extends through the holes with a clearance such as to allow modification of the inclination of the jaws 3, 4 and 7 with respect to the connecting rod.

Advantageously, the area (more precisely, the diameter) of the hole 18 of the jaw 3 is larger than the area of the holes 18 of the jaws 4 and 7.

According to some embodiments, the hole 18 of the jaw 3 has an area from 200 mm² (in particular, 225 mm²) to 300 mm² (in particular, 275 mm²). Alternatively or additionally, the holes 18 of the jaws 4 and 7 have, each independently of the other, an area from 100 mm² (in particular, 125 mm²) to 200 mm² (in particular, 175 mm²). Alternatively or additionally, the rod 17 has cross section with an area from 80 mm² (in particular, 100 mm²) to 140 mm² (in particular, 120 mm²). It should be noted that the rod 17 (since it has to pass through the holes 18) has cross section with an area smaller than the holes 18 (at least in the area of said holes 18).

Advantageously, the hole 18 of the jaw 3 has within it an abutment element (of per se known type and not illustrated), which is adapted to prevent the movement of the elastic element 12 towards the jaw 4. According to some embodiments, the cited abutment element (which has an annular shape) is arranged in the area of the end of the hole 18 of the jaw 3 facing the jaw 4. In this way, a sort of housing in the hole of the jaw 3 is defined, and inside said housing the elastic element 12 (more precisely, a part of the elastic element 12) is kept in a substantially fixed position.

Advantageously, the supporting element 11 has a supporting surface facing (in particular, adapted to come into contact with) the jaw 3 with an area smaller than the areas of the surfaces 4b and 7a which come into contact with each other. In particular, the areas of the surfaces 4b and 7a which come into contact with each other are at least one and a half times the area of the cited supporting surface.

According to some embodiments, the cited supporting surface has an area from approximately 290 mm² (in particular, from 300 mm²) to approximately 340 mm² (in particular, 320 mm²). Alternatively or additionally, areas of the surfaces 4b and 7a which come into contact with each other have, each one independently of the other, an area from 500 mm² (in particular, 520 mm²) to 550 mm² (in particular, 540 mm²) (not considering the surface development of the projections 15 and of the seats 16).

The characteristics described above make transfer of the force from the clamp 2 to the clamp 6 particularly efficient so that the locking surfaces 20 of the jaws 7 and 8 move closer to each other and the ends 21 of the jaws 7 and 8 move farther away from each other.

Advantageously, each locking surface 20 has a first concave (rounded) portion and a second inclined portion arranged between the first portion and the rod 17. The second portion of the locking surface 20 of the jaw 3 extends from the respective first portion towards the jaw 4. The second portion of the locking surface 20 of the jaw 4 extends from the respective first portion towards the jaw 3. The second portion of the locking surface 20 of the jaw 7 extends from the respective first portion towards the jaw 8. The second portion of the locking surface 20 of the jaw 8 extends from the respective first portion towards the jaw 7.

It should be noted that the particular shape of the surfaces 20 makes adaptation of the seats 5 and 10 to support elements (for example, screws and/or bars) of different thickness particularly effective.

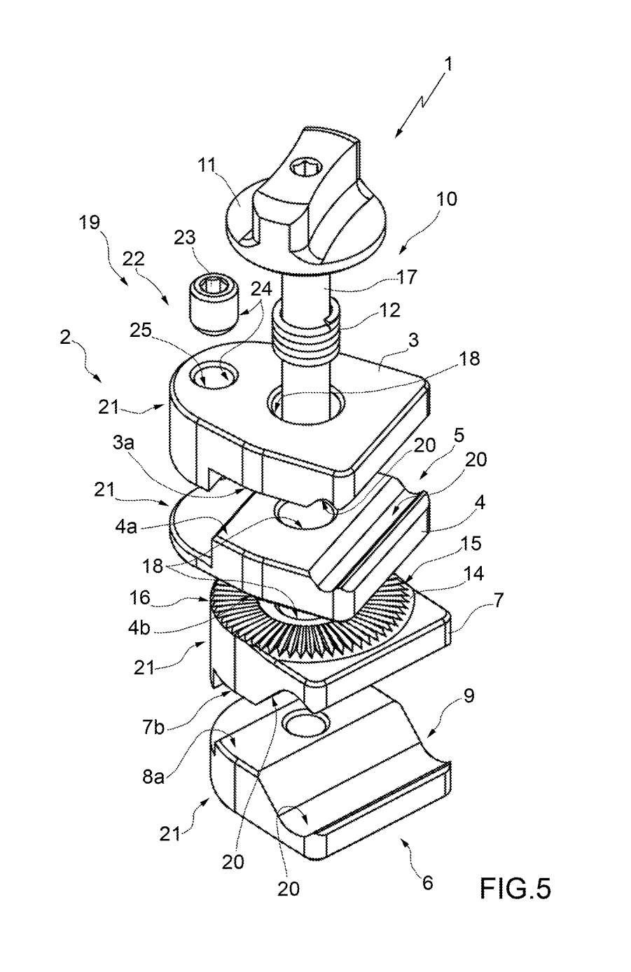

The embodiment of the system 1 illustrated in FIG. 5 is substantially identical to the embodiment of FIG. 4 and differs from it only due to the fact that the seat 5 is adapted to house a screw instead of a bar.

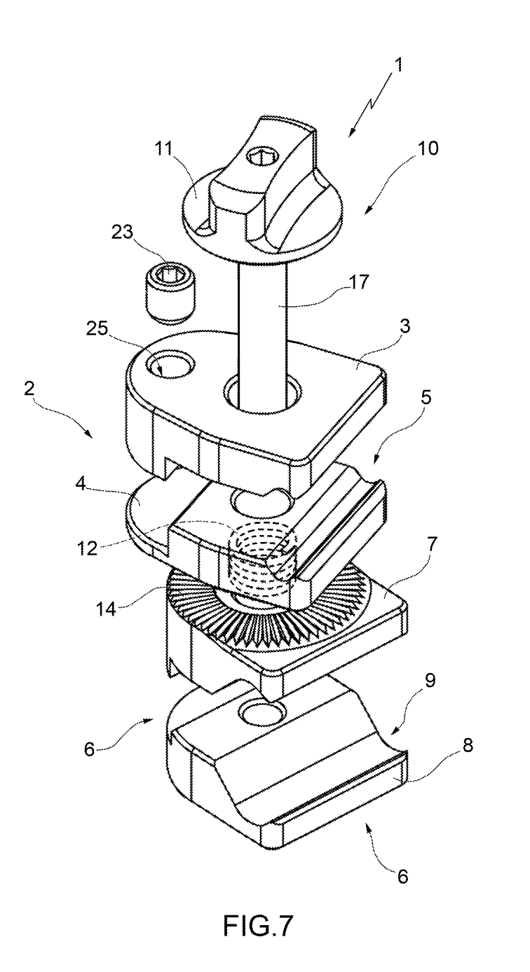

The embodiment of the system 1 illustrated in FIG. 7 is substantially identical to the embodiment of FIG. 5 and differs from it only due to the fact that the elastic element 12 is arranged between the clamps 2 and 6 instead of between the clamp 2 and the supporting element 11. In other words, the system 1 of FIG. 7 has the closing device 19 as described above, but a different locking assembly 11 (in particular, it has no parts in common with it and is adapted to be activated separately from it).

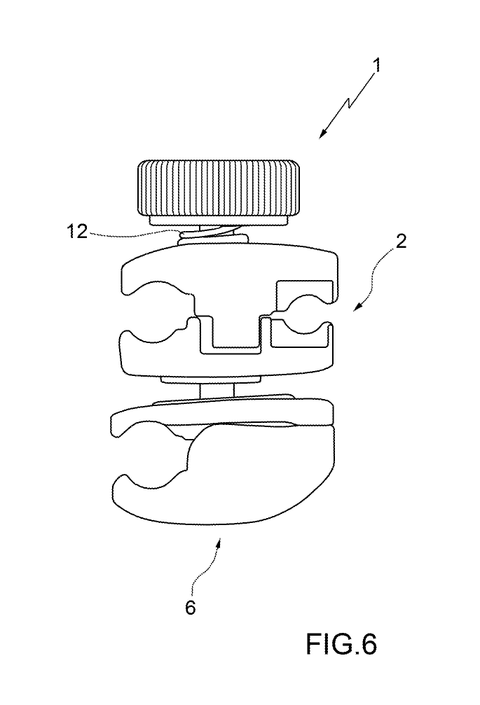

The embodiment of the system 1 illustrated in FIG. 6 differs from the embodiment of FIG. 4 due to the fact that it has another closing device. However, the system 1 of FIG. 6 comprises a locking assembly 10 as described above.

1. An external fixing system for an orthopaedic exoskeleton, having at least a first and a second support element; the fixing system comprising:
a first clamp (2), having a first jaw and a second jaw (3, 4) and having a first clamping seat (5), adapted to house the first support element;
a second clamp (6), adjacent and connected to the first clamp (2) in an orientable manner, the second clamp having a third jaw and a fourth jaw (7, 8) and a having second clamping seat, adapted to house the second support element; and
a locking assembly (10) for limiting the relative orientation of the first clamp and the second clamp (2, 6) relative to one another;
wherein the locking assembly (10) comprises:
a supporting element (11) positioned on the opposite side of the first clamp (2) relative to the second clamp (6) and is connected to the second clamp (6); and
an elastic element (12) positioned between the supporting element (11) and the first clamp (2), thereby exerting a force upon the first clamp (2) towards the second clamp;
wherein an elastic means, which in resting conditions allow the first clamp and the second clamp (2, 6) to be kept at a distance, is absent between the first clamp and the second clamp (2,6).

2. A fixing system according to claim 1, wherein the locking assembly (10) comprises at least a first locking element (13) located on the first clamp (2); and a second locking element (14) located on the second clamp (6) and is adapted to be coupled to the first locking element (13) so as to limit the relative rotation of the first clamp and the second clamp (2, 6) relative to one another.

3. A fixing system according to claim 2, wherein the first locking element and the second locking element (13, 14) have respective shapes that are substantially complementary to each other.

4. A fixing system according to claim 2, wherein at least one between the first locking element and the second locking element (13, 14) has at least one projection; at least the other one between the first locking element and the second locking element (13, 14) has at least one seat engagable by the projection to limit the relative rotation of the first clamp and the second clamp (2, 6) relative to each other.

5. A fixing system according to claim 1, further comprising a connecting rod (17), connected to the supporting element (11) and to the second clamp (6) and extends through the first clamp (2); the first clamp (2) being at least partially able to slide along the connecting rod (17).

6. A fixing system according to claim 5, wherein the locking assembly (10) comprises a coupling system between the connecting rod (17) and the second clamp (6), said coupling system being adapted to normally keep the distance between the supporting element (11) and the second clamp (6) fixed and being able to be activated to vary said distance.

7. A fixing system according to claim 1, wherein the elastic element (12) comprises a spring, which is arranged between the supporting element (11) and the first clamp (2); in particular, the spring being arranged around said connecting rod (17).

8. A fixing system according to claim 1, wherein the second clamp (6) is connected to the first clamp (2) in a rotary manner; and the locking assembly (10), once engaged, locks the relative rotation of the first clamp and the second clamp (2, 6) with respect to each other.

9. A fixing system according to claim 1, further comprising a closing device (19) to vary the width of the first and the second clamping seat (5, 9) by changing the relative orientation of the first jaw and the second jaw (3, 4) and of the third jaw and the fourth jaw (7, 8), respectively.

10. A fixing system according to claim 9, wherein each jaw (3, 4, 7, 8) has a respective locking surface (20); the locking surfaces (20) of the first jaw and the second jaw (3, 4) at least partially delimit the first clamping seat (5); the locking surfaces (20) of the third jaw and the fourth jaw (7, 8) at least partially delimit the second clamping seat (9); wherein each jaw (3, 4, 7, 8) has an end (21), which is opposite to the respective locking surface (20); and the closing device (19) comprises at least one thrust assembly (22), that exerts a thrust to push apart said ends (21) of the first jaw and the second jaw (3, 4), thereby bringing the locking surfaces (20) of the first jaw and the second jaw (3, 4) closer together.

11. A fixing system according to claim 10, wherein the thrust assembly (22) comprises at least a thrust element (23), located in the area of at least one of said ends (21) and biasing apart said ends (21) of the first jaw and the second jaw (3, 4).

12. A fixing system according to claim, wherein the first clamp and the second clamp (2, 6) are arranged and shaped so that the pushing-apart thrust exerted by the thrust assembly (22) is transferred to the second clamp (6) in order that a force is exerted upon the third jaw (7) in such a way that the locking surfaces (20) of the third jaw and the fourth jaw (7, 8) move closer to one another and said ends (21) of the third and the fourth jaw (7, 8) move away from one another.

13. A fixing system according to claim 1, wherein the elastic element (12) is arranged in contact between the supporting element (11) and the first clamp (2) to exert a force on the first clamp (2) towards the second clamp.

14. A fixing system according to claim 12, wherein the second jaw (4) has a first coupling surface (4b) and the third jaw (7) has a second coupling surface (7a); wherein the second coupling surface (7a) faces the first coupling surface (4b); and the supporting element (11) has a support surface facing the first jaw (3) and has an area that is smaller than the areas of the first coupling surface and the second coupling surface (4b, 7a) that come into contact with each another.

15. A fixing system according to claim 1, wherein the locking assembly (10) locks the relative orientation of the first clamp and the second clamp (2, 6) relative to one another.

16. A fixing system according to claim 4, wherein the first locking element and the second locking element (13, 14) each have a plurality of projections and a plurality of seats engagable by the projections.

17. A fixing system according to claim 4, wherein more in the first locking element and the second locking element (13, 14) are knurled.
